(19) United States
(12) Patent Application Publication
(10) Pub. No.: US 2004/0172672 A1
(43) Pub. Date: Sep. 2, 2004

(54) Method for producing transgenic monocotyledonous plants

(76) Inventors: Theresa C. Wilkinson, Champaign, IL (US); Henry T. Wilkinson, Champaign, IL (US); Richard E. Wagner, Bloomington, IN (US)

Correspondence Address: ICE MILLER, ONE AMERICAN SQUARE, BOX 82001, INDIANAPOLIS, IN, 46282, US

(21) Appl. No.: 10/477,059

(22) PCT Filed: May 15, 2002
(86) PCT No.: PCT/US02/15380

(30) Foreign Application Priority Data
May 15, 2001 (US) 60291128

(51) Int. Cl. A01H 1/00; C12N 15/82

(52) U.S. Cl. 800/278; 435/468

(57) ABSTRACT

Methods for transforming monocot plants are provided. The method comprise the steps of excising an embryo from a seed of the plant, allowing the embryo to develop to a stage highly receptive for transformation, transforming the developing embryo, and developing the embryo into a plantlet, wherein the transformation occurs without passing through a callus phase.

### FIELD OF THE INVENTION

This method relates to a method for producing transformed monocots from excised mature embryos without the need for a callus phase during the transformation, or selection process, thus precluding or minimizing somoclonal variation. In particular, this invention relates to development of nonchimeric transgenic monocots by directly transforming the meristematic cells of the apical dome of excised embryos at a specific stage during embryo germination.

### BACKGROUND OF THE INVENTION

The development of methods to transform plants with genes from other species has presented unlimited opportunities to create plants with unique and superior agronomic and horticultural traits, as well as specialty traits, such as the production of antibiotics. At least 120 plant species have been stably transformed following incorporation of a transgene into the nuclear genome. (Birch, R. G. 1997. Plant transformation: problems and strategies for practical application. Annu. Rev. Plant Physiol. Plant Mol. Biol. 48:297-326). In 1997, the percentage of acres planted to transgenic cotton, soybean, and corn was approximately 25, 18, and 10% respectively (James, C. 1997. Global status of transgenic crops in 1997, ISAAA Briefs No. 5. ISAAA: Ithaca, N.Y.). Most of these transgenic crops contained one of three genes: cp4 epsps, which confers tolerance to glyphosate, bar, which confers tolerance to glufosinate, and bt which confers resistance to insects. As well as nuclear transformation, transgenes also have been stably incorporated into the genomes of chloroplasts and mitochondria. (Hutchison R, R. A. Roffey and R. T. Sayre. 1996. Chloroplast transformation. pp. 180-196. In: Molecular Genetics of Photosynthesis, Frontiers in Molecular Biology. Arderson B, Salter A H, and Barber J, eds. Oxford University Press).

Successful transformation of plants demands that certain criteria be met. (Hansen, G. and M. S. Wright. 1999. Recent advances in the transformation of plants. Trends in Plant Science 4: 226-231; Birch, R. G. 1997. Plant transformation: problems and strategies for practical application. Annu. Rev. Plant Physiol. Plant Mol. Biol. 48:297-326). Among the requirements for transformation are: target tissues that are competent for propagation and regeneration; the ability to recover fertile transgenic plants at a reasonable frequency; and a simple, efficient, reproducible, genotype-independent, and cost-effective process.

Methods used to deliver a transgene to plants include but are not limited to transformation systems using: (1) Agrobacterium (Hiei, Y. et al, U.S. Pat. No. 5,591,616; Hooykass, P. J. J. and R. A. Schilperoort. 1992. Agrobacterium and plant genetic engineering. Plant Mol. Biol. 19:15-38; Tepfer, D. 1990. Genetic transformation using Agrobacterium rhizogenes. Physiol. Plant 79: 140-146; Zupan, J. R. and P. Zambryski. 1995. Transfer of T-DNA from Agrobacterium to the plant cell. Plant Physiol. 107:1041-1047); (2) direct gene transfer into protoplasts by liposomes (Gad, A. E., N. Rosenberg and A. Altman. 1990. Liposome-mediated gene delivery into plant cells. Physiol. Plant 79:177-183), or by chemical treatment or electroporation (Kryzyzek, R. et al, U.S. Pat. No. 5,472,869; Negrutiu, I., J. Dewulf, M. Pietrzak, J. Botterman, E. Rietveld. 1990 Hybrid genes in the analysis of transformation conditions. II. Transient expression vs. sable transformation: analysis of parameters influencing gene expression levels and transformation efficiency. Physiol. Plant. 79: 197-205); (3) direct gene transfer into tissue using biolistics (Christou, P. 1992. Genetic transformation of crop plants using microprojectile bombardment. The Plant Journal 2:275-281; Sanford, J. C., F. D. Smith and J. A. Russell. 1993. Optimizing the biolistic process for biological applications. Methods in Enzymology 217: 483-509; Birch, R. G. and T. Franks. 1991. Development and optimization of microprojectile systems for plant genetic transformation. Austrian J. Plant Physiol. 18:453-469); (4) silicon carbide whiskers (Kaeppler, H. F., D. A. Sommers, H. W. Rines and A. F. Cockburn. 1992. Silicon carbide fiber-mediated transformation of plant cells. Theo. Appl. Genet 84:560-566; Thompson, J. A., P. R. Drayton, B. R. Frame, K. Wang, and J. M. Dunwell 1995. Maize transformation utilizing silicon carbide whiskers—a review. Euphytica 85:75-80); and (5) microinjection (Neuhaus, G. and G. Spangenburg. 1990. Plant transformation by microinjection techniques. Physiol. Plant. 79:213-217).

The two most widely used techniques are Agrobacterium mediated transformation and biolistics. A limitation of Agrobacterium mediated transformation is that the host range is strain dependent. While initially it was assumed that the host range was limited to dicotyledenous plants, progress has been made in developing strains that successfully transform monocotyledons, albeit with much effort. Monocotyledonous plants transformed using an Agrobacterium-mediated gene delivery system include rice and turfgass. (Hiei Y., S. Ohta, T. Komari, and T. Kumashiro. 1994. Efficient transformation of rice (Oryza sativa L.) mediated by Agrobacterium and sequence analysis of the boundaries of the T-DNA. Plant J. 6:271-282). In contrast, biolistic transformation is genotype independent and transformation of most monocot species is routinely achieved using the biolistic process. (Vasil, 1. 1994 Molecular Improvement of Cereals. Plant Molecular Biology 25:925-937; Christou, P. 1995. Strategies for variety-independent genetic transformation of important cereals, legumes and woody species utilizing particle bombardment. Euphytica 85:13-27).

Plant cells from organized tissues have the capacity to dedifferentiate to form a mass of dividing unorganized tissue referred to as callus. With few exceptions most transformation systems use a callus phase. Calli can be produced in vitro by placing plant parts, referred to as explants, on solid growth media amended with hormones. Callus growth can be maintained indefinitely, or calli may be used give rise to whole plants via somatic embryos, a process referred to as totipotency. Each step of regeneration from callus to a whole plant requires adjustments in the type and concentration of hormones added to the growth media.

Somatic embryos are suitable targets for transformation because they are nonchimeric (putatively arising from a single cell), prolific, and easily maintained, manipulated, and transformed. A limitation of using callus is that not all species, or even cultivars within a species, have the capacity to produce regenerable calli. Because the reasons for this species and genotype recalcitrance are not fully understood, a great deal of time and money are spent empirically developing tissue culture protocols. In many of the agronomic species, it is often the elite cultivars that are recalcitrant and the obsolete cultivars that the most cooperative. As a result, calli from nonelite cultivars are often initially used to move a transgene into the species. Following regeneration, the transgenic plant is used as a donor in a conventional breeding program. Of course, this process adds years to the time it takes to develop a commercially acceptable transgenic cultivar. It is clear that the callus phase is the rate-limiting step in a transformation system.

Plants regenerated from callus can be phenotypically and genotypically different from the parent plant. This introduction of spontaneous and heritable genetic changes during tissue culture, referred to as somoclonal variation, has been reported for many species. (Karp, A. 1994. Origins, causes and uses of variation in plant tissue cultures. In: Vasil, I. K., Thorpe, T. A. (eds) Plant cell and tissue culture. Kluwer Academic Publishers. Dordrecht, The Netherlands, pp 139-151). Somoclonal variation has been used as a source of genetic variation for plant improvement. (Duncan, R. R. 1996. Tissue culture induced variation and crop improvement. Advances in Agronomy 58:2201-204; Evans, D. A. 1989. Somoclonal variation: genetic basis and breeding applications. Genetics 5:46-50; Karp A. 1995. Somoclonal variation as a tool for crop improvement. Euphytica 85:295-302). For example, Msikita and Wilkinson (1994. Powdery mildew resistance in Kentucky bluegrass regenerated from excised seed pieces. Euphytica 78:199-205) developed a system for increasing the genetic resistance to disease of both Kentucky bluegrass (Poa pratensis L.) and creeping bentgrass (Agrostis palustris L) by selection of somoclonal variants produced in tissue culture. While this approach was successful in selecting for disease resistance, this technique also resulted in other morphological and physiological changes from that of the original mother plant. This problem has also been noted by other researchers. (Larkin, P. J. and W. R. Scowcroft. 1981, Theor. Appl. Genet. 60, 197-214). Furthermore, the effects of somoclonal variation are often deleterious, and may include loss of viability, albinism, and sterility. In addition, the magnitude of somoclonal variation may be underestimated due to a comparison of relatively few traits, such as height and yield. Cowen et al. (Cowen, N. M., S. A. Thompson and T. C. Wilkinson. 1990. Culture associated variation in maize inbreds. Plant Breeding 104:134-143) and Wilkinson and Thompson (Wilkinson, T. C. and S. A. Thompson. 1987. Genotype, medium and genotype x medium effects on the establishment of regenerable maize callus. Maydica 32:89-105) reported that 87% of a corn inbreed line exhibited variation when several traits, including leaf size, were measured.

The goal of a transformation system is to transfer a gene from one organism to another. To evaluate the performance of the transgene, it is imperative that genetic variation between the genetically modified organism and its parent be minimized. Expression of the transgene can be affected by numerous factors, such as the compatibility of the construct with the organism or the site of insertion in the genome. (Gruber, M. Y. and W. L. Cosby. 1993. Vectors for plant transformation. In: Glick, B. R. and J. E. Thompson eds. 1993. Methods in Plant Molecular Biology and Biotechnology. CRC Press. Baco Raton, Fla.; Bregitzer, P., S. E. Halkbert and P. G. Lemaux. 1998. Somoclonal variation in the progeny of transgenic barley. Theor. Appl Genet. 96:421-425).

Because of the high incidence of somoclonal variation, there is little guarantee that a regenerated transgenic plant would be identical to the parent source of the explant. As a result, transgenic plants often require extensive field-testing, not only to evaluate the performance of the transgene but also to ensure that agronomic traits that comprise the elite cultivar have not been changed. Often, the transgenic plant is used as a donor parent for backcrossing in a breeding program.

One way to reduce somoclonal variation is to minimize the time the tissue remains as callus. Hence, new callus is often used for transformation. However, this requires more extensive manipulation of the target tissue than simply maintaining and subdividing existing callus. Furthermore, a brief callus phase, even when using meristematic region for target tissue, still does not guarantee that somoclonal variation will not occur. Somoclonal variation has been reported even when in vitro growth is limited to 4 weeks (Devaux, P. A. Kilian, and A. Kleinhofs. 1993. Anther culture and Horedeum bulbosum-derived barley double haploids: mutations and methylation. Mol. Gen. Genet. 241:674-679). The need to reduce such random genetic change may become the overriding consideration in the choice of tissue culture and gene transfer systems (Birch, R. G. 1997. Plant transformation: problems and strategies for practical application. Annu. Rev. Plant Physiol. Plant Mol. Biol. 48:297-326).

Christou and McCabe (U.S. Pat. No. 5,989,915) offer another approach that minimizes the callus phase, in which meristems or immature zygotic embryos are directly transformed by biolistics followed by a brief callus phase on hormone amended media to promote shoot and root formation. This method has been successful for soybean, bean, cotton, peanut, wheat, and rice. (Christou, P., T. L. Ford, and M. Kofron. 1992. The development of a variety independent gene-transfer method for rice. Trends Biotechnol. 10:239-247; Wan, Y., P. G. Lemaux. 1994. Generation of large numbers of independently transformed fertile barley plants. Plant Physiol. 104:337-48; Weeks, T. J., O. D. Anderson, A. E. Blechl. 1993. Rapid production of multiple independent lines of fertile transgenic wheat (Triticum aestivum). Plant Physiol. 102:1077-1084; Christou, P. 1995. Strategies for variety-independent genetic transformation of important cereals, legumes and woody species utilizing particle bombardment. Euphytica 85:13-27; Russell, D. R., K. M. Wallace, J. H. Bathe, B. J. Martinell, D. E. McCabe. 1993. Stable transformation of Phaseolus vulgaris via electric-discharge mediated particle acceleration. Plant Cell Rep. 12:165-169; Gammbley, R. L., J. D. Bryant, N. P. Masel, and G. R. Smith. Cytokinin enhanced regeneration of plants from microprojectile bombarded sugarcane meristematic tissue. Aust. J. Plant Physiol. 21:603-612; Brar, G. S., B. A. Cohen, C. L. Vick and G. W. Johnson. 1994. Recovery of transgenic peanut (Arachis hypogea L.) plants from elite cultivars utilizing ACCELL R technology. Plant Journal 5: 745-753). Zygotic embryos are embryos that develop from the zygote, formed following the union of gametes or sex cells (Phillips, G. C. and O. L. Gamborg. 1995. Plant Cell, Tissue and Organ Culture: Fundamental Methods. Springer. 358 p.; Esau, K. 1977. Embryo and Seedling. Chapter 24 In: Anatomy of Seed Plants, 2nd ed., John Wiley and Sons, New York. 550 p). Zhong and Stricklen (U.S. Pat. No. 5,767,368) teach a similar method for producing transgenic corn by transforming meristem primordia using biolistics. Likewise, Bowen et al (U.S. Pat. No. 5,736,369) teach a similar method for transforming maize by bombarding immature embryos. Both techniques require a culture phase on media amended with hormones for shoot and root formation. In addition to the requirement for a callus culture phase, the transformation system of Christou and McCabe (U.S. Pat. No. 5,989,915) results in a high incidence of chimerical plants. The high incidence of chimeras results from the inability to deliver DNA accurately to the progenitor cells in the apical meristem. In most dicots and some monocots, the shoot apical meristem includes three superimposed layers: a superficial L1, a subsurface L2 and a deeper L3. (Sussex, I. M. 1989. Developmental programming of the shoot meristem. Cell 56:225-229). The L1 layer gives rise exclusively to the epidermis, whereas the tissues that arise from the L2 and L3 layers are position dependent. Approximately three cells, referred to as initials, give rise to the cells of each layer. Christou and McCabe (U.S. Pat. No. 5,830,728) teach a method for selecting nonchimeric transgenic plants produced using the transformation system described in U.S. Pat. No. 5,989,915.

The development of nonchimeric transgenic plants using a completely tissue-culture free transformation system has not been achieved using biolistics for either monocots or dicots. Christou et al (Christou, P., T. L. Ford, and M. Kofron. 1992. The development of a variety independent gene-transfer method for rice. Trends Biotechnol. 10:239-247) report that attempts to develop transgenic rice plants using biolistics to transform meristems of germinating rice seedling was unsuccessful, due to the interference from the coleoptile, a sheath-like structure that encloses the epicotyl. In most grasses the coleoptile is a hollow cone with an opening near the apex, which allows the shoot to emerge during germination. Lee and Berg (U.S. Pat. No. 5,948,956) teach a method for producing transgenic turfgrasses through the direct transformation of nodal segments using microprojectile bombardment, Agrobacterium mediated genetic transfer, or electroporation, although the authors only report on biolistics. Lee and Berg claim a hormone-free medium for regenerating St. Augustine, which is purportedly unique among the turfgrasses in that nodal explants do not require additional shoot- or root-inducing hormones. This technique resulted in a transformation rate of 0.2% using the bar gene as a selectable marker. No information was provided regarding whether the regenerated plants where chimeral or clonal.

Agrobacterium-mediated transformation systems have been employed to develop transgenic monocot plants. However, no methods for monocots have been developed which completely lack a callus phase or hormone amended media. Hiei and Komari (U.S. Pat. No. 5,591,616) and Dong et al (Dong, J., W. Teng, W. G. Buchholtz and T. C. Hall. 1996. Agrobacterium-mediated transformation of Javanica rice. Molec. Breed. 2:267-276) report on a method of transforming rice using calli. Dong and Teng (U.S. Pat. No. 6,037,522) teach a method for minimizing the length of time in culture by transforming excised inflorescences of rice followed by a short regeneration period in culture. Smith et al (U.S. Pat. No. 5,164,310) teach a method for transforming excised shoot meristems and apical buds of wheat and corn, as well as several dicots. The transformed explants required a culture phase with hormone-amended media. No mention was made if the regenerated plants were chimerical or clonal. Goldman and Graves (U.S. Pat. No. 6,020,539) teach a method for transforming seedling of Graminae by wounding followed by cultivation with Agrobacterium tumefaciens. Bechtold et al (1993. In planta Agrobacterium mediated gene transfer by infiltration of adult Arabidopsis plants. C. R. Acad. Sci. Paris, Life Sciences 316:1194-1199) report a tissue-culture-free method of producing transformed seed of Arabidopsis thaliana, a dicot, following vacuum infiltration of infloresences with Agrobacterium. No similar system has been reported for monocots.

### SUMMARY OF THE INVENTION

The present disclosure is directed to the production of transgenic monocotyledonous plants using a variety of transformations systems without the need for a callus phase, thus minimizing or precluding somoclonal variation. This is accomplished by excising embryos from germinating seeds, allowing development to a specific stage in which the meristematic cells of the apical dome are highly receptive to transformation, and then transforming the target cells. In an illustrated embodiment, following transformation, plant development continues on nutrient media that is not amended with hormones or vitamins. Identification of transformed plants is accomplished following transfer of the young seedling to nutrient media amended with a selective agent, such as an herbicide or antibiotic.

Accordingly, in one aspect, the invention provides a method for producing transgenic plants without the need for a callus phase.

In another aspect, the invention provides a method for transforming a monocot plant, said method comprising the steps of excising an embryo from a seed of the plant, placing the excised embryo on media not amended with growth hormones, allowing the embryo to develop to a stage highly receptive for transformation, transforming the developing embryo, transferring the transformed embryo to a hormone-free selective media that supports growth of the embryo, and developing the embryo into a plantlet.

In yet another aspect, the invention provides a method for producing nonchimeric turfgrass plants following transformation of meristematic cells of apical domes from excised embryos.

In still another aspect, the invention provides a population of turfgrass plants according to the methods described herein.

The foregoing merely summarizes certain aspects of the present invention and is not intended, nor should it be construed, as limiting in any manner.

All patents and other publications referenced herein are hereby incorporated by reference in their entirety.

### BRIEF DESCRIPTION OF DRAWINGS

FIG. 1 shows an excised apical dome of mature Poa pratensis seed. Apical dome (6 hrs after excision) viewed from the side, with excised surface on the upper left and opposite to the apex of the dome. Apical dome=0.75 mm wide.

FIG. 2 shows an excised apical dome of mature Poa pratensis seed: non-transformed. Apical dome (48 hrs after excision) viewed from the top. The excised surface can be seen at the right of the apical dome as a milky-white convex area. The emerging embryo (white) can be seen growing at the left. Apical dome=0.75 mm wide; emerging embryo=0.5 mm long.

FIG. 3 shows an excised apical dome of mature Poa pratensis seed: transformed. Apical dome (48 hrs after excision) viewed from the side. The excised surface can be seen at the left of the apical dome as a translucent, convex area. The emerging embryo (top) has been stained to visualize GUS gene expression. Apical dome=0.75 mm wide; emerging embryo=0.2 mm wide.

FIG. 4 shows a germinating, transformed seedling of Poa pratensis. The apical dome (72 hrs after excision) can be seen at the top; emerging from it is the embryo (1.5 mm long); and an emerging root extends 4 mm from the apical dome (lower left). No callus was formed.

### DETAILED DESCRIPTION OF THE INVENTION

The present invention provides methods of transforming plants without using a callus phase, whereby the meristematic cells of the emerging apical dome from excised embryos are targeted for transformation at a specific time when they are highly receptive for transformation. In a preferred embodiment, all progeny cells from the transformed meristematic cell contribute to a producing a transgenic clonal plant (i.e. one that is not chimeric). In an illustrated embodiment, the plants are monocots, particularly monocots of the family Poaceae.

The embryo of a mature seed will germinate and produce a sexually viable plant given sufficient nutrients and proper growing conditions. Following imbibition of the embryo, cell division, i.e., growth will commence. The subsequent ontogeny of the embryo includes apical dome or apex development; organization and initiation of surface meristems and tissues; and the subsequent development of shots and roots, i.e., a seedling. It is during the early stages of embryo germination, particularly apical dome formation, that genetic transformation may be achieved without interrupting normal development and without undifferentiated growth (callus).

Grass species differ in the exact pattern of embryogenic development, but share many similarities in the general pattern of development. The mature embryo of a seed includes a mother cell(s) from which other cells originate. The position of the mother cell is specie dependent and will change in terms of its relative proximity to the apical dome surface. As additional cells are formed by the mother cell, the new cells will become organized and differentiated, giving rise to preliminary tissues. These preliminary tissues will then differentiate to form the roots and shoots. Transformation as describe herein comprises the introduction of DNA into differentiating or meristematic cells. In one embodiment that achieves efficient transformation, the introduction of DNA is timed to coincide with both active development and accessibility of the mother cell.

The stage of development when the emerging domes are transformed affects the progenitor cells of the meristem. By optimizing the stage of development during which transformation takes place, transformation can occur at a high rate, and the incidence of chimerical plants may be reduced. As used herein, the term “highly receptive” refers to this optimal time for transformation. Highly receptive embryos are embryos that can be transformed at a high rate and produce chimerical plants at a low rate. The incubation time to reach this highly receptive state varies by species, and even by cultivar. However, the incubation time needed to reach the highly receptive stage can be determined empirically.

As illustrated, the process for transforming apical domes may be divided into 4 steps: (1) preparation of the embryos; (2) transformation of the apical, dome; (3) selection of transformed plants; and (4) seed production and evaluation of progeny for nonchimeric lines.

1. Preparation of Embryos.

In one embodiment, mature seeds (150-200 grams) are soaked in distilled water for 12 hours at room temperature. The water is decanted and replaced with a surface disinfesting solution (10% Clorox bleach, 50% ethanol (95% solution) and 40% distilled sterile water). The seeds are submerged in 50 ml of the disinfesting solution and stirred vigorously for 10 minutes. The seeds are allowed to settle to the bottom of the vessel and the sterilization solution is decanted. The seeds are rinsed five times with a 100× volume of sterile distilled water. After the final decantation, the seeds are transferred to a sterile container. It is understood that other protocols may be employed to provide seeds of sufficient viability and disinfection.

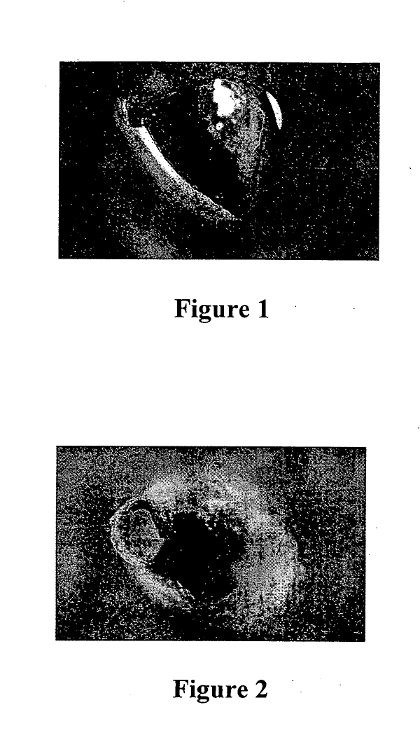

The seed coats are removed, illustratively by pressing on the top of a seed with a needle and peeling off the seed coat using a scalpel. The embryo is separated from the endosperm by an excision, preferably perpendicular to the long axis of the seed and as close to the embryo as possible (within 1 mm of the embryogenic tissue is preferred) (see FIG. 1). The excised embryo is immediately placed excision side down on a medium such as ¼ strength Murashige & Skoog (MS) medium (1 g/L MS salts (Sigma), 7.5 g/L sucrose, pH 5.7, 30.0-g/L agar). In an illustrated embodiment, the media is not supplemented with hormones or vitamins or is essentially free of growth hormones. It is understood that a medium should be chosen such that callus formation will not occur. The embryos are incubated until they become highly receptive, illustratively for up to 28 hours under fluorescent light (16 hr) at 22 C (see FIG. 2). The embryos are observed every six-hour during incubation. Embryos that show both germination and emergence, visible as a single apical dome are selected for transformation. The timing of dome emergence after placement on MS medium can vary depending on the temperature, species, and natural variation in seed germination among populations of seeds, illustratively between 6-44 hr after placement on MS medium. For Kentucky bluegrass and annual bluegrass maximum dome emergence occurs at 28 and 24 hrs, respectively after plating (see Experiment 2, Test A). The selected embryos are transferred to fresh ¼ strength MS medium and oriented as above with the developing apical dome facing away from the media.

2. Transformation of the Apical Dome from Germinating Embryos.

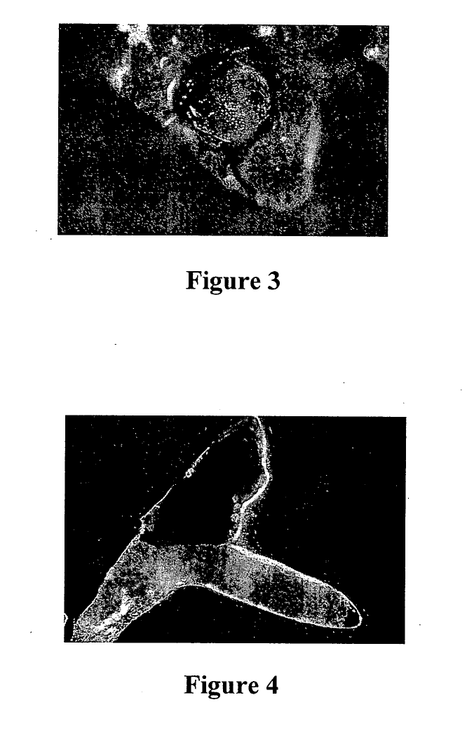

Emergent domes from germinating embryos can be transformed with a transgene using a variety of transformation methods including but not limited to biolistics, Agrobacterium-mediated gene transfer, silicon-fiber whiskers, electroporation, and vacuum infiltration. These techniques are known to those skilled in the art. In the illustrated embodiment, DNA is delivered to the apical dome using a biolistic process. Biolistics have been described in detail in other publications and the technique is commonly used in plant transformation, as are the other techniques listed above. Briefly, DNA is coated on a metal carrier, layered on a support and accelerated using helium gas. The DNA-coated particles penetrate the target tissue. The DNA becomes stably incorporated into the nuclear genome by methods not yet fully understood. Variables include helium pressure, distance of the target tissue from the support, tissue age, target tissue used, DNA concentration, microprojectile concentration, and duration of blast. Methods for maximizing the effectiveness for biolistics have been published and should be familiar to those skilled in the art. A variety of particle acceleration devices have been developed. (See, for example, U.S. Pat. Nos. 5,015,580; 5,036,006; 4,945,050; 5,478744, 6,004,287; 5,141,131; 5,478,744; 5,371,015; 5,179,022; 5,100,792; 5,204,253). In the illustrative embodiment, a particle inflow gun as described by Finer (Finer et al.1992. Development of the particle inflow gun for DNA delivery to plant cells. Plant Cell Reports 11: 323-3280) is used. A brief description of the materials and methods for delivery of DNA to the emergent domes from the germinating embryos using the particle inflow gun is as follows: 5 μl of plasmid DNA (1 μg/μl) was mixed with 35 μl tungsten (60mg in 500 μl 100% ethanol), 50 μl CaCl₂ and 20 μl spermidine, vortexed, incubated for 10 minutes at room temperature, washed in 100% ethanol and resuspended in 50 μl of 100% ethanol. Five μl of the DNA-tungsten suspension was loaded onto the screen surface. A helium gas at 72 psi accelerated the particles for 0.5 seconds in a vacuum chamber at 28-30 mg Hg. The tissue was placed 9 cm from the orifice of the macro syringe. A transformed embryo is shown in FIG. 3. It is understood that variation of the methods and devices used for transformation is within the scope of this invention.

Genes used to transform the plants include but are not limited to those conferring herbicide tolerance, diseases resistance, insect resistance, and stress tolerance. The constructs have appropriate untranscribed and untranslated leader and termination sequences required for gene expression in monocots, as is known in the art. (Vasil, 1. 1994. Plant Molecular Biology 25: 925-937). Components required for gene expression in monocots include, but are not limited to: promoters, such as ubil (Christensen, A. H. and P. H. Quail. 1996. Ubiquitin promoter based vectors for high-level expression of selectable and or screenable marker genes in monocotyledonous plants. Transgenic Research 5:213-218), CaMV 35S promoter (Kat el al. 1987 Science 236:1299-1302; Fraley et al. 1996. U.S. Pat. No. 5,530,196), actin (McElroy et al. 1991.;Mol. Gen. Genetics 231: 150-16); nontranslated 5′ elements, such as the rice actin intron (McElroy et al. 1991. Mol. Gen. Genet. 231:150-160); marker genes, such as the nptII and hph, which confer resistance to the antibiotics kanamycin and hygromycin, respectively, or bar and cp4, which confer tolerance to the herbicides glufosinate and glyphosate, respectively; and a terminator, such as the nos termination sequence. The construct pAHC25 is used herein for the illustrative embodiment. For a complete description of pAHC25, see Christensen, A. H. and P. H. Quail. 1996. Ubiquitin promoter based vectors for high-level expression of selectable and or screenable marker genes in monocotyledonous plants. Transgenic Research 5:213-218. The construct contains two genes, bar (phosphinothricin acetyl transferase gene from Streptomyces hygroscopicus), and uida (β-glucoronidase) used in GUS assays. Transcription of the bar gene is regulated by the maize ubiquitin promoter (Ubi-1) and the NOS terminator from Agrobacterium tumefaciens T-DNA.

3. Post Transformation Culturing and Selection.

In the illustrated embodiment, the transformed embryos are allowed to grow on the MS medium following transformation for 4 days and then transferred to MS medium amended with selective agents, such as antibiotics or herbicides. In the case of selection for the bar gene, the medium was amended with Finale™ (glufosinate-ammonium) to a concentration of 3 μg/ml (active ingredient). After 2-4 weeks the surviving plants are transferred to fresh medium amended with 3 μg/ml glufosinate-ammonium and incubated at room temperature and 16 hr of light for an additional month. Other selective markers are known in the art and may be used within the scope of this invention. The resulting transformed seedlings are transplanted to the soil and grown in a greenhouse. A germinating seedling is shown in FIG. 4.

4. Seed Production and Evaluation.

In the illustrated embodiment, putative transformed plants are sprayed in the greenhouse with ammonium-glufosinate. Tissue from glufosinate tolerant plants are analyzed for the presence of the bar gene by PCR and Southern blot hybridization. Seeds from the transformed plants are collected and assayed for the presence of the introduced genes. After transformation is confirmed, seed production may take place as is known in the art.

### EXPERIMENT I

### Germination of Excised Embryos

Two variants of Poa pratensis and annual bluegrass are studied to determine the optimum stage for transformation for each of these variants. The results of the study on germinating embryos of the cultivars “Midnight” and “Ascot” of Poa pratensis compared to annual bluegrass (Poa annua) are presented in Table 1. Table 1 shows that the two variants of Poa pratensis and the one variant of Poa annua may be transformed at about 28 hours of incubation, with the two variants of Poa pratensis allowing for more variation in the timing of transformation.

TABLE 1
Development of Kentucky bluegrass (Poa pratensis) and Annual
bluegrass (Poa annua) seedlings from excised, mature embryos

| | No. of germinated | | | Total no. of |
|---|---|---|---|---|
| No. of | embryos after | | | germinating |
| excised Embryos | 28 hr | 32 hr | 48 hr | embryos |
| P. pratensis | | | | |
| var. ‘Midnight’ | | | | |
| 50 | 25 | 12 | 7 | 44 |
| 50 | 33 | 7 | 7 | 47 |
| 45 | 23 | 11 | 11 | 45 |
| 60 | 40 | 15 | 5 | 60 |
| 50 | 26 | 9 | 5 | 40 |
| P. pratensis | | | | |
| var. ‘Ascot’ | | | | |
| 50 | 40 | 0 | 0 | 40 |
| 50 | 36 | 7 | 3 | 46 |
| 30 | 26 | 4 | 0 | 30 |
| 30 | 22 | 8 | 0 | 30 |
| 45 | 28 | 0 | 12 | 40 |
| [Poa annua | | | | |
| var. annua] | | | | |
| 50 | 50 | 0 | 0 | 50 |
| 50 | 50 | 0 | 0 | 50 |
| 50 | 50 | 0 | 0 | 50 |
| 50 | 50 | 0 | 0 | 50 |
| 45 | 45 | 0 | 0 | 45 |

### EXPERIMENT 2

### Transient Expression of Transformed Embryos

To determine the efficiency of the transformation system, germinating embryos of the cultivar Midnight were transformed 24 hours after excision and placed on media as described above. GUS gene expression was determined 24 hours after on media as described above. GUS gene expression was determined 24 hours after transformation following standard procedures that are familiar to those skilled in the art (Jefferson, R. A. 1987. Assaying chimeric genes in plants. The GUS gene fusion system. Plant Molec. Biol. Rep. 5:387-405). The results of two separate experiments are presented in Table 2.

TABLE 2
Frequency of mature Kentucky bluegrass variety ‘Midnight’
embryos staining positive for expression of GUS

| | No. of germinating Embryos | No. of embryos testing positive |
|---|---|---|
| Trial | Treated with GUS gene and | for GUS gene expression after |
| No. | Promoter (pAHC25) | 24 hr |
| Test A | | |
| 1 | 5 | 3 |
| 2 | 4 | 3 |
| 3 | 5 | 1 |
| 4 | 4 | 1 |
| 5 | 4 | 3 |
| 6 | 4 | 3 |
| 7 | 4 | 0 |
| 8 | 5 | 1 |
| 9 | 4 | 2 |
| Test B | | |
| 1 | 4 | 4 |
| 2 | 5 | 2 |
| 3 | 4 | 3 |
| 4 | 4 | 2 |
| 5 | 5 | 5 |
| 6 | 5 | 1 |
| 7 | 4 | 1 |

### EXPERIMENT 3

### Transformation of Kentucky Bluegrass Domes with the Bar Gene

Emerging embryos of Kentucky bluegrass cvs. Midnight and Ascot were transformed as described above with the bar gene. Following selection on media amended with glufosinate, plantlets were transferred to soil and grown in the greenhouse. The leaf tips of 15 putative transgenic plants were painted with glufosinate (Finale™) at a concentration of 200-250 μg/ml of the active ingredient. Plants were observed over a 1-week period. Some plants developed discoloration and drying of treated leaves. Five plants developed no symptoms and remained healthy, indicating transformation.

1. A method for transforming a monocot plant, said method comprising the steps of:
excising an embryo from a seed of the plant,
placing the excised embryo on media not amended with growth hormones,
allowing the embryo to develop to a stage highly receptive for transformation,
transforming the developing embryo,
transferring the transformed embryo to a hormone-free selective media that supports growth of the embryo, and
developing the embryo into a plantlet.

2. The method in claim 1 wherein the plant that is a member of the family Poaceae.

3. The method of claim 2 wherein the plant is Poa pratensis.

4. The method of claim 3 wherein the allowing the embryo to develop step comprises incubating the embryo for 24 to 28 hours prior to the transforming step.

5. The method of claim 2 wherein the allowing the embryo to develop step comprises incubating the embryo for 24 to 48 hours prior to the transforming step.

6. The method in claim 1 wherein the transforming the developing embryo step comprises a transformation technique selected from the group consisting of biolistic, Agrobacterium, electroporation, microinjection, and whiskers.

7. The method of claim 1 wherein the embryo is transformed using a vector comprising a selective marker.

8. The method of claim 7 wherein the selective marker comprises a gene for conferring herbicide tolerance.

9. The method of claim 8 wherein the herbicide is selected from the group consisting of glyphosate and glufosinate, and the method further comprises the step of
exposing the plantlet to the herbicide.

10. The method of claim 8 wherein the vector comprises a second gene selected from the group consisting of herbicide tolerance, insect resistance, disease resistance, environmental tolerance, growth regulation, and developmental control.

11. The method of claim 7 wherein the selective marker comprises a gene conferring antibiotic resistance.

12. The method of claim 11 wherein the antibiotic is selected from the group consisting of hph and nptII, and the method further comprising the step of growing the embryo on a medium amended with the antibiotic.

13. The method of claim 1 further comprising the step of-producing progeny of the plantlet that express a gene introduced during the transforming step.

14. A plant produced according to the method of claim 1.

15. A method for transforming a monocot plant, said method comprising the steps of:
excising an embryo from a seed of the plant,
allowing the embryo to develop to a stage highly receptive for transformation,
transforming the developing embryo, and
developing the embryo into a plantlet,
wherein the allowing, transforming, and developing steps all occur without passing through a callus phase.

16. A method for transforming a monocot plant, said method comprising the steps of:
excising an embryo from a seed of the plant,
placing the excised embryo on media essentially free of growth hormones,
allowing the embryo to develop to a stage highly receptive for transformation,
transforming the developing embryo,
transferring the transformed embryo to a selective medium that supports growth of the embryo essentially free of growth hormones, and
developing the embryo into a plantlet.
